(19) United States
(12) Patent Application Publication
(10) Pub. No.: US 2021/0257123 A1
(43) Pub. Date: Aug. 19, 2021

(54) X-RAY COLLIMATOR ASSEMBLY AND X-RAY SOURCE ASSEMBLY

(71) Applicant: OUR UNITED CORPORATION, Xi'an City (CN)

(72) Inventor: Hao YAN, Xi'an City (CN)

(21) Appl. No.: 17/142,846

(22) Filed: Jan. 6, 2021

(30) Foreign Application Priority Data
Feb. 17, 2020 (CN) 202010097702.1

(51) Int. Cl. G21K 1/04; A61B 6/06; A61B 6/00

(52) U.S. Cl. CPC G21K 1/04; A61B 6/4035; A61B 6/06

(57) ABSTRACT

Embodiments of the present disclosure provide an X-ray collimator assembly and an X-ray source assembly. The X-ray collimator assembly includes a light limiter, a light filter, and a housing, the light limiter and the light filter are arranged in the housing, the housing includes an X-ray incident end and an X-ray emitting end; the light limiter is arranged between the X-ray incident end and the X-ray emitting end of the housing; and the light filter is arranged between the X-ray incident end and the light limiter, or the light filter is arranged between the X-ray emitting end and the light limiter. The X-ray collimator assembly provided by the embodiments of the present disclosure can effectively reduce noise interferences with the human tissue image obtained by X-ray projection, such that the projection image is more accurate.

### CROSS-REFERENCE TO RELATED APPLICATIONS

The present application claims priority to Chinese application No. 202010097702.1, filed on Feb. 17, 2020, and entitled “X-RAY COLLIMATOR ASSEMBLY AND X-RAY SOURCE ASSEMBLY”, of which are hereby incorporated by reference in its entireties.

### TECHNICAL FIELD

Embodiments of the present disclosure relate to the field of X-rays, and in particular to, an X-ray collimator assembly.

### BACKGROUND

In medical image detection, applications of checking human tissues using various rays are increasingly widely used. When the structure of human tissues is checked using various rays, e.g., X-rays (Roentgen rays), different black and white images can be formed on a screen or X-ray film. During human tissue imaging using an existing X-ray imaging apparatus, due to different thicknesses of different parts of a human body, a projection image obtained by X-rays emitted from an X-ray source through different parts of the human body has a large noise and poor image quality.

### SUMMARY

In view of this, one of the technical problems to be solved by the present disclosure is to provide an X-ray collimator assembly, thereby avoiding the problems of large noise and poor image quality of X-ray projection imaging caused by different thicknesses of different parts of the human body.

An embodiment of the present disclosure provides an X-ray collimator assembly, including: a light limiter, at least one light filter, and a housing;

where the light limiter and the light filter are arranged in the housing, and the housing includes an X-ray incident end and an X-ray emitting end;

the light limiter is arranged between the X-ray incident end and the X-ray emitting end; and

the light filter is arranged between the X-ray incident end and the light limiter; or the light filter is arranged between the X-ray emitting end and the light limiter.

Alternatively, in an embodiment of the present disclosure, the light filter includes at least two light filters corresponding to different irradiated object sections.

Alternatively, in an embodiment of the present disclosure, the X-ray collimator assembly further includes a base configured to fix positions of the at least two light filters corresponding to different irradiated object sections in the housing, and switch the light filters corresponding to different irradiated object sections through the base.

Alternatively, in an embodiment of the present disclosure, the base is configured to switch the light filters corresponding to different irradiated object sections in a push-pull manner.

Alternatively, in an embodiment of the present disclosure, the light filter is a light filter having different light filtering areas, and the different light filtering areas correspond to different irradiated object sections.

Alternatively, in an embodiment of the present disclosure, the light filter is made of an opaque heavy metal compound, or consists of a transparent matrix and heavy metal compound particles doped in the transparent matrix.

The present disclosure further discloses an X-ray source assembly, including: a bulb tube, a light limiter, and at least one light filter; where

the light filter is located between the bulb tube and the light limiter, or the light filter is located on one side of the light limiter away from the bulb tube; and

both the light filter and the light limiter are located on a path of an X-ray beam generated by the bulb tube.

Alternatively, in an embodiment of the present disclosure, there are at least two light filters, the light filters have different curvatures and correspond to different body parts of a to-be-detected object, and the body parts have different thicknesses in an incident direction of X-rays.

Alternatively, in an embodiment of the present disclosure, the light filter has a plurality of light filtering areas, the light filtering areas have different curvatures and correspond to different body parts of the to-be-detected object, and the body parts have different thicknesses in the incident direction of the X-rays.

Alternatively, in an embodiment on an X-ray source assembly of the present disclosure, the light filter includes a light filter having different light filtering areas, and the different light filtering areas correspond to different irradiated object sections.

Alternatively, in an embodiment of the present disclosure, the light filter includes at least two light filters corresponding to different irradiated object sections.

Alternatively, in an embodiment on an X-ray source assembly of the present disclosure, the X-ray source assembly further includes a base configured to fix positions of the at least two light filters corresponding to different irradiated object sections, and switch the light filters corresponding to different irradiated object sections through the base.

Alternatively, in an embodiment on an X-ray source assembly of the present disclosure, the base is configured to switch the light filters corresponding to different irradiated object sections in a push-pull manner.

Alternatively, in an embodiment on an X-ray source assembly of the present disclosure, the light filter is made of an opaque heavy metal compound, or consists of a transparent matrix and heavy metal compound particles doped in the transparent matrix.

Alternatively, in an embodiment on an X-ray source assembly of the present disclosure, the X-ray source assembly further includes a mounting structure, and the light filter is fixed between the light limiter and the bulb tube through the mounting structure, or the light filter is fixed to one side of the light limiter away from the bulb tube through the mounting structure.

Alternatively, in an embodiment on an X-ray source assembly of the present disclosure, the X-ray source assembly includes at least two light filters corresponding to different irradiated object sections.

Alternatively, in an embodiment on an X-ray source assembly of the present disclosure, the mounting structure is located between the light limiter and the bulb tube, and the mounting structure has a first end matching an output end of the bulb tube, and/or the mounting structure has a second end matching an input end of the light limiter, or the mounting structure has no connection with the bulb tube and/or the light limiter.

Alternatively, in an embodiment on an X-ray source assembly of the present disclosure, thickness of the mounting structure on the path of the X-ray beam is less than a preset threshold.

Alternatively, in an embodiment on an X-ray source assembly of the present disclosure, the mounting structure is located on one side of the light limiter away from the bulb tube, and the mounting structure has a third end matching an output end of the light limiter, or the mounting structure has no connection with the light limiter.

An embodiment of the present disclosure provides an X-ray collimator assembly, including a light limiter, a light filter, and a housing, the light limiter and the light filter are arranged within the housing, the housing is provided with an X-ray incident end and an X-ray emitting end; the light limiter is arranged between the X-ray incident end and the X-ray emitting end of the housing; and the light filter is arranged between the X-ray incident end and the light limiter, or the light filter is arranged between the X-ray emitting end and the light limiter of the housing. In the embodiments of the present disclosure, a light filter is additionally provided in the X-ray collimator assembly to compensate for the effects of different thicknesses of a human body on the quality of X-ray images. The X-ray collimator assembly provided in the embodiments of the present disclosure can effectively reduce noise interferences with a human tissue image obtained by direct projection with X-rays, such that the projection image is clearer and more accurate.

### BRIEF DESCRIPTION OF THE DRAWINGS

FIG. 1 is a schematic structural diagram of an X-ray collimator assembly provided in an embodiment of the present disclosure;

FIG. 2 is a schematic structural diagram of a light filter provided in an embodiment of the present disclosure;

FIG. 3 is a schematic structural diagram of another light filter provided in an embodiment of the present disclosure;

FIG. 4 is a schematic diagram of a human tissue image obtained by X-ray projection provided in an embodiment of the present disclosure;

FIG. 5 is a schematic structural diagram of a base and at least two light filters provided in an embodiment of the present disclosure; and

FIG. 6 is a schematic structural diagram of an X-ray source assembly provided in an embodiment of the present disclosure.

### DETAILED DESCRIPTION

Specific implementations of embodiments of the present disclosure will be further described below with reference to the accompanying drawings of the embodiments of the present disclosure.

### Embodiment I

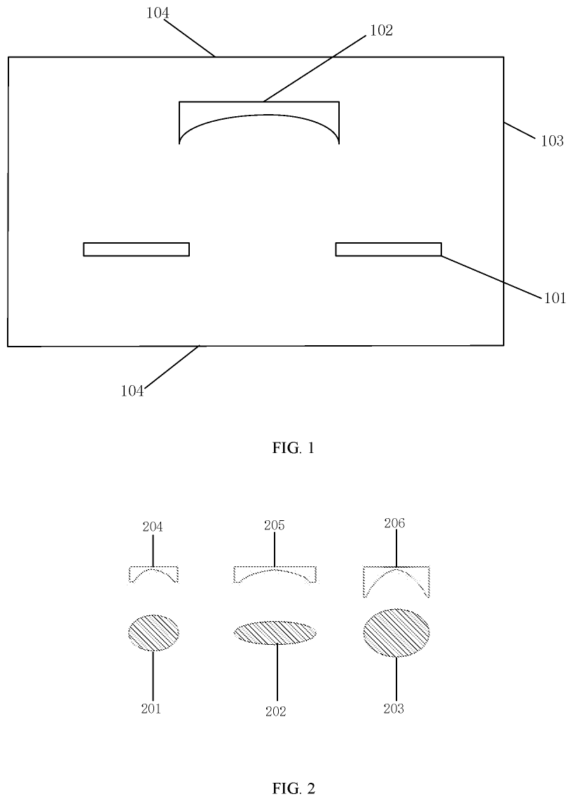

FIG. 1 is a schematic structural diagram of an X-ray collimator assembly provided in an embodiment of the present disclosure. As shown in FIG. 1, this embodiment provides a schematic structural diagram of an X-ray collimator assembly, including: a light limiter 101, at least one light filter 102, and a housing 103;

where the light limiter 101 and the light filter 102 are arranged in the housing 103, and the housing 103 includes an X-ray incident end 104 and an X-ray emitting end 105;

the light limiter 101 is arranged between the X-ray incident end and the X-ray emitting end of the housing 103; and

the light filter 102 is arranged between the X-ray incident end and the light limiter of the housing 103; or the light filter 102 is arranged between the X-ray emitting end 104 and the light limiter 101 of the housing 103.

Here, the light limiter 101 is described for an X-ray collimator assembly of X-rays:

In this embodiment, the light limiter 101 includes at least two light limiting blades, and a gap between the at least two light limiting blades forms a light-passing hole through which the X-rays can pass.

In the embodiment of the present disclosure, the at least two light limiting blades may be mutually separated or move together to adjust a size of the light-passing hole formed by the at least two light limiting blades, thereby adjusting a size of a projection area of X-rays projected onto a to-be-detected object. Therefore, the at least two light limiting blades according to the present disclosure can both be made of a material having a shielding effect on X-rays.

In an implementation, the housing 103 is further provided with a control structure connected to the at least two light limiting blades, and positions of the at least two light limiting blades are moved through the control structure, thereby adjusting the size of the light-passing hole formed by the at least two light limiting blades.

When the X-rays are used for detection, because the to-be-detected object is of an uncertain size, and the X-rays are also a kind of rays harmful to biological tissues, it is necessary to minimize the projection area of the X-rays projected onto the to-be-detected object, to prevent unnecessary X-ray dosage from damaging the to-be-detected object (e.g., a human tissue). In this case, a control structure configured to adjust a position of the light limiter 101 may be further provided on the housing. The control structure can adjust the size of the light-passing hole on the light limiter by adjusting the position of the light limiter 101, thereby adjusting the size of the projection area of the X-rays.

Regarding the light filter 102 in the X-ray collimator assembly provided in the present disclosure, two specific examples are provided here for description:

Alternatively, in a first example of the present disclosure, the light filter includes at least two light filters corresponding to different irradiated object sections.

When a projection image is acquired by projecting a to-be-detected object using X-rays, as shown in FIG. 2, FIG. 2 is a schematic diagram of a human tissue image projected with X-rays provided in an embodiment of the present disclosure, and the to-be-detected object is human tissues. As shown in FIG. 2, 201 denotes a section shape of a human head, 202 denotes a section shape of a human chest, and 203 denotes a section shape of a human torso. The human head, chest, and other torso have different thicknesses along a radiation direction of rays. If only one light filter is used to adjust the X-rays, the obtained projection image cannot reflect accurate image information of the to-be-detected object with different thicknesses. Therefore, in the embodiment of the present disclosure, three light filters corresponding to different object sections may be provided based on thicknesses of the to-be-detected object in different areas, and the object section is a section of the object parallel to an incident direction of the X-rays. For example, for a light filter 204, a light filter 205, and a light filter 206, when the human head 201 is detected, the light filter 204 is used to adjust a projection intensity of an X-ray beam; when the human chest 202 is detected, the light filter 205 is used to adjust a projection intensity of an X-ray beam; and when the human torso 203 is detected, the light filter 206 is used to adjust a projection intensity of an X-ray beam, such that the projection images obtained after projecting the X-ray beams onto the to-be-detected objects with different thicknesses are more accurate.

Cross sections of the light filter 204, the light filter 205, and the light filter 206 are all of a shape that is thin in the middle and thick on both sides. Each of the light filters has a constant curvature of curved surface. The three light filters have different curvatures of curved surface from each other. The light filters with different curvatures correspond to the to-be-detected objects with different thicknesses. A user can adjust a light filter corresponding to a current to-be-detected object based on different thicknesses of the to-be-detected object, such that the X-ray beam has better projection effects through different to-be-detected objects, thereby improving the quality of the projection images.

Alternatively, in a second example of the present disclosure, the light filter 102 may also be a light filter having different light filtering areas, and the different light filtering areas correspond to different irradiated object sections.

When the X-rays are used to project a human body to obtain a human tissue image, distances of different areas of the to-be-detected object from an X-ray source are different, and incident angles of the X-rays when the X-ray beams are projected onto the to-be-detected object are different, such that intensities of the X-rays projected onto different positions of the to-be-detected object will be different, and the obtained projection images contain much noise information and poor image quality. In this case, the light filter is set to a shape that is thin in the middle and thick on the edge to adjust the intensities of X-ray projection and the incident angles of the X-rays in different projection areas, such that a projection image of a to-be-detected area away from the X-rays is clearer. In the embodiment of the present disclosure, the light filter is set as a light filter with different light filtering areas, which are used to adjust the intensities of the X-rays projected onto the to-be-detected object, such that the obtained projection image is clearer.

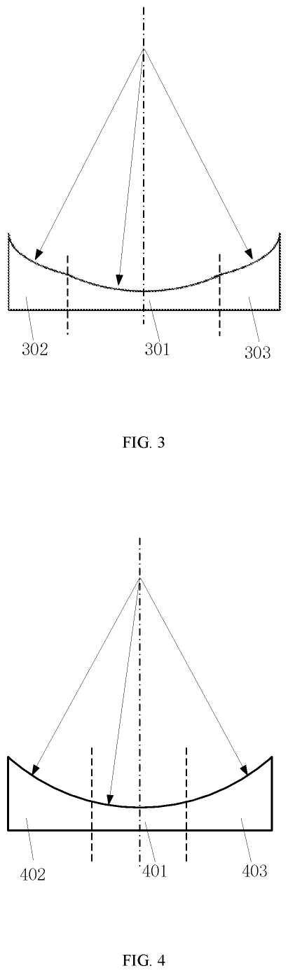

In an implementation of the present disclosure, the light filter 102 is a filter including at least two different light filtering areas, each of the at least two light filtering areas is connected to an adjacent light filtering area, and each light filtering area has different X-ray compensation dosages. Here, an example is provided to describe a structure of the light filter 102 including at least two different light filtering areas in the present disclosure. As shown in FIG. 3, FIG. 3 is a light filter 30 provided in an embodiment of the present disclosure. The light filter 30 includes three light filtering areas, namely a light filtering area 301, a light filtering area 302, and a light filtering area 303. A center of a curvature of curved surface of the light filtering area 301, a center of a curvature of curved surface of the light filtering area 302, and a center of a curvature of curved surface of the light filtering area 303 are located on a given straight line or overlapped, where the curvature of curved surface of the light filtering area 301 is a first curvature used to detect a human head; the curvature of curved surface of the light filtering area 302 is a second curvature, and the curvature of curved surface of the light filtering area 303 of the light filter is a third curvature. The second curvature may be identical to or different from the third curvature. When the second curvature is identical to the third curvature, the light filtering area 302 and the light filtering area 303 are configured to detect parts other than the human head, e.g., a chest, while when the second curvature is different from the third curvature, the light filtering area 303 can be configured to detect parts other than the human head and chest. Alternatively, the structure of the light filter and the corresponding X-ray compensation dosage may be set as required. This is not limited in the present disclosure. It should be noted that the curvature of curved surface is used to indicate a degree of curvature of a curved surface. For example, the curvature of curved surface may be an average of principal curvatures of a plurality of points on a curved surface, or an average of Gaussian curvatures of a plurality of points. Of course, here only example description is provided, which does not mean that the present disclosure is limited to this.

Alternatively, in an implementation of the present disclosure, different light filtering areas of a light filter may also have a given curvature. As shown in FIG. 4, FIG. 4 is a schematic structural diagram of a light filter provided in this embodiment. A curvature of a light filtering area 401, a curvature of a light filtering area 402, and a curvature of a light filtering area 403 are all identical, but a volume of the light filtering area 401, a volume of the light filtering area 402, and a volume of the light filtering area 403 exposed to different X-ray radiation fields are different, such that the light filtering area 401, the light filtering area 402, and the light filtering area 403 have different X-ray compensation dosages. Here, merely example description of the structure of the light filter of the present disclosure is provided in the present disclosure, which does not mean that the present disclosure is limited to this. In an implementation of the present disclosure, the curved surface of the light filter may also be set to have one or more light filtering areas of different curvatures, or have one or more light filtering areas of different X-ray compensation dosages. This is not limited in the present disclosure.

Alternatively, in an embodiment of the present disclosure, the X-ray collimator assembly further includes a base configured to fix positions of the at least two light filters corresponding to different irradiated object sections in a housing, and switch the light filters corresponding to different irradiated object sections through the base.

The base can switch the light filters corresponding to different irradiated object sections in a push-pull manner.

In an implementation of the present disclosure, an opening structure may also be provided on the housing, and the base can be pulled out of the housing through the opening structure on the housing. When it is necessary to replace and load the light filter, the base is pulled out the through the opening structure to replace and load the light filter fixed on the base. After completing the replacement, the base is pushed into the housing through the opening structure on the housing, such that the base and the housing are relatively fixed to complete switching the light filter. The opening structure is provided on the housing, such that the base can be pulled out of the opening structure, which not only makes the replacement of the light filter simpler and easy to operate, but also facilitates the inspection and maintenance of the X-ray source assembly.

In addition, since X-rays are an invisible light, the X-rays, when being projected onto a human body, will have certain killing effects on human cells. Therefore, when the X-rays are used to detect human tissues, it is necessary to simulate a light field outputted by the X-rays. Generally, an X-ray imaging system includes a light field simulating unit, which includes at least one visible light generating mechanism, and is located on one side of the light limiter close to a bulb tube on a path of an X-ray beam to simulate a light path of the X-ray beam using a visible light generated by the light field simulating unit. When the light limiter according to this embodiment is used, since the light filter will shield the visible light, light field simulation may be implemented by pulling the base out of the housing to facilitate the passage of the visible light.

The above approach is only an implementation on light filter switching in this embodiment. In an embodiment of the present disclosure, there may also be other settings. For example, the base may be set as a disc-shaped base, which is provided with a plurality of clamping slots configured to fix light filters of different types, and is eccentrically arranged inside the housing. The housing is provided with a control structure that can rotate the disc. When it is necessary to replace the light filter, the disc is rotated through the control structure to adjust a relative position of the light filter fixed on the disc and the housing, such that the required light filter is located on an X-ray path, and the light filter switching can be completed. Of course, here, merely example description of different light filter switching approaches is provided, which does not mean that the present disclosure is limited to this. Similarly, in order to facilitate the light field simulation, the disc-shaped base may also be provided with a vacancy without loading a light filter.

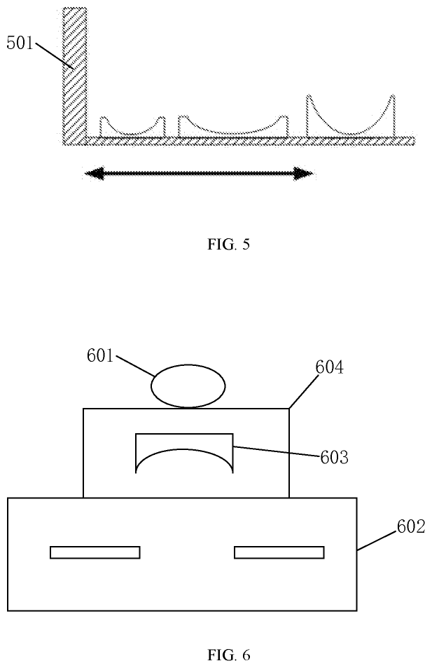

In an implementation of the present disclosure, as shown in FIG. 5, FIG. 5 is a schematic structural diagram of a base and at least two light filters provided in this embodiment, including a drawer-type base 501, and three light filters with different section shapes loaded on the base 501. Based on thickness requirements of different irradiated objects, a position of the drawer-type base 501 can be adjusted in a push-pull manner, such that a required light filter is located on an X-ray path. For different to-be-detected objects, a light filter matching a to-be-detected object is used to improve the image definition of projection imaging with an X-ray beam. Similarly, in order to facilitate light field simulation, the drawer-type base may also be provided with a vacancy without loading a light filter.

In an implementation of the present disclosure, an opening structure or a clamping slot may be provided at a position where the light filter and the base are connected, the light filter may be provided at the opening structure of the base, and the light filter is fixed relative to the base through the clamping slot to facilitate the maintenance and replacement of the light filter when the light filter is damaged. Of course, here, only an example is provided to describe a correction relationship between the light limiter and the base, which does not mean that the present disclosure is limited to this.

In an embodiment of the present disclosure, the light filter is made of an opaque heavy metal compound, or made of a transparent matrix and heavy metal compound particles doped in the transparent matrix. The transparent matrix is used to facilitate the light field simulation.

The X-ray collimator assembly disclosed in this embodiment includes a light limiter, a light filter, and a housing. The light limiter and the light filter are arranged in the housing, the housing includes an X-ray incident end and an X-ray emitting end, the light limiter is arranged between the X-ray incident end and the X-ray emitting end of the housing, and the light filter is arranged between the X-ray incident end and the light limiter, or the light filter is arranged between the X-ray emitting end and the light limiter. The X-ray collimator assembly provided in this embodiment can be used to adjust an area of an X-ray projection area through the light limiter, and adjust an intensity of X-rays projected onto a to-be-detected object through different light filtering area distributions on the light filter, such that the projection images projected through the X-ray collimator assembly are clearer.

### Embodiment II

The present disclosure further provides an X-ray source assembly. As show in FIG. 6, FIG. 6 is a schematic structural diagram of an X-ray source assembly provided in an embodiment of the present disclosure, including a bulb tube 601, a light limiter 602, and at least one light filter 603;

the light filter 603 is located between the light limiter 602 and the bulb tube 601, or the light filter 603 is located on one side of the light limiter 602 away from the bulb tube 601; and

both the light filter 603 and the light limiter 602 are located on a path of an X-ray beam generated by the bulb tube.

Alternatively, in an implementation on the X-ray source assembly in this embodiment, the light filter includes a light filter having different light filtering areas, and the different light filtering areas correspond to different irradiated object sections.

Alternatively, in an implementation on the X-ray source assembly in this embodiment, the light filter includes at least two light filters corresponding to different irradiated object sections.

Alternatively, in an implementation on the X-ray source assembly in this embodiment, the X-ray source assembly further includes a base configured to fix positions of the at least two light filters corresponding to different irradiated object sections, and switch the light filters corresponding to different irradiated object sections through the base.

Alternatively, in an implementation on the X-ray source assembly in this embodiment, the base is configured to switch the light filters corresponding to different irradiated object sections in a push-pull manner.

Here, an example is provided to describe a setting of the base. The base may be an L-shaped drawer-type base. The base is provided with a clamping slot adapted to a shape of the light filter. The light filter may be fixed on the base through the clamping slot. When it is necessary to replace a light filter arranged on the base and corresponding to an irradiated object due to different irradiation requirements, the L-shaped base is pushed or pulled to move a position of the base relative to the bulb tube, such that the light filter is located on the path of the X-ray beam, thereby completing the replacement of different light filters. Of course, here only one approach of switching different light filters is provided in this embodiment, and there may also be other setting types of bases to switch the light filters. For example, the base may be set as a disc-shaped base. The disc-shaped base is provided with a plurality of clamping slots configured to fix light filters of different types, and is eccentrically arranged in a work area of a radiation field of an X-ray source. When it is necessary to replace the light filter, the disc is rotated through the control structure to adjust a relative position of the light filter fixed on the disc and the housing, such that the light filter is located on the path of the X-ray beam, and the light filter switching can be completed. Of course, those skilled in the art may also design other forms of light filter switching approaches based on actual conditions. This is not limited in the present disclosure.

Alternatively, in an implementation on the X-ray source assembly in this embodiment, the light filter is made of an opaque heavy metal compound, or consists of a transparent matrix and heavy metal compound particles doped in the transparent matrix.

Alternatively, in an implementation on the X-ray source assembly in this embodiment, the X-ray source assembly further includes a mounting structure 604, and the light filter 603 is fixed between the light limiter 602 and the bulb tube 601 through the mounting structure 604, or the light filter 603 is fixed to one side of the light limiter 602 away from the bulb tube 601 through the mounting structure 604.

Alternatively, in an implementation on the X-ray source assembly in this embodiment, the mounting structure 604 is located between the light limiter 602 and the bulb tube 601, and the mounting structure 604 has a first end matching an output end of the bulb tube, and/or the mounting structure 604 has a second end matching an input end of the light limiter 602, or the mounting structure 604 has no connection with the bulb tube 601 and/or the light limiter 602.

Alternatively, in an implementation on the X-ray source assembly in this embodiment, thickness of the mounting structure on the path of the X-ray beam is less than a preset threshold.

In an X-ray imaging system, the bulb tube is generally used as an X-ray generating source. An existing device generally performs projection imaging on the to-be-detected object directly by connecting the bulb tube and the light limiter. However, due to different distances of different to-be-detected areas on the to-be-detected object from the bulb tube, intensities of X-rays projected to different positions of the to-be-detected object are different, and projection angles of the X-rays at different positions of the to-be-detected object are different, resulting in poor quality and much noise of the final X-ray image. Without changing an existing X-ray imaging system, the X-ray source assembly provided in the embodiment of the present disclosure adjusts intensities and incident angles of the X-rays generated by the bulb tube, such that the image of the to-be-detected object is of high quality and is clear with little noise.

Alternatively, in an implementation of this embodiment, the mounting structure 604 is located on one side of the light limiter 602 away from the bulb tube 601, and the mounting structure 604 has a third end matching an output end of the light limiter 602, or the mounting structure 604 has no connection with the light limiter 602.

For example, in a first implementation of the present disclosure, the mounting structure 604 may be a housing, a cavity for accommodating a light filter is formed inside the housing, and a connection structure matching the light filter is provided on the cavity inside the housing. The connection structure may be a clamping slot or other connection structures, and the light filter is fixed inside the housing through the connection structure. The housing is fixed between a bulb tube and a light limiter, and the clamping slot on the housing is located on a path of an X-ray beam, such that the light filter fixed on the clamping slot is located on the path of the X-ray beam generated by the bulb tube, thereby adjusting the projection intensity of the X-rays using the light filter.

When the housing is provided between the light limiter 602 and the bulb tube 601, the housing has the first end matching the output end of the bulb tube 601, and/or the housing has the second end matching the input end of the light limiter 602, or the housing has no connection with the bulb tube 601 and/or the light limiter 602.

When the housing is located on one side of the light limiter 602 away from the bulb tube 601, the housing has the third end matching the output end of the light limiter 602, or the housing has no connection with the light limiter 602.

The X-ray source assembly provided in this embodiment includes a bulb tube, a light filter, and a light limiter. The light filter is located between the bulb tube and the light limiter, or the light filter is located on one side of the light limiter away from the bulb tube. The X-ray source assembly disclosed in this embodiment can adjust the X-ray projection distribution intensity without changing the existing X-ray imaging system, and improve the image definition of X-ray imaging.

In the embodiments of the present disclosure, the description is provided with reference to the drawings. However, some embodiments may not use one or more of these specific details, or use one or more of these specific details in combination with other known methods and structures. In the above description, numerous specific details, such as specific structures, dimensions, and processes, are described to provide a thorough understanding of the present disclosure. Reference to “one or an embodiment” throughout the present specification means that a particular feature, structure, or characteristic described in the embodiment is included in at least one embodiment of the present disclosure. Therefore, the appearances of the phrase “in one or an embodiment” in various places in the present specification are not necessarily all referring to the same embodiment. In addition, the specific features, structures, configurations, or characteristics may be combined in one or more embodiments in any suitable manner.

The above description merely provides embodiments of the present disclosure, and is not intended to limit the present disclosure. For those skilled in the art, the present disclosure may have various modifications and alterations. Any modification, equivalent replacement, improvement, and the like made within the spirit and principles of the present disclosure should be included within the scope of the appended claims of the present disclosure.

What is claimed is:

1. An X-ray collimator assembly, comprising: an X-ray collimator, at least one X-ray filter, and a housing;
wherein the collimator and the filter are arranged in the housing, and the housing includes an X-ray incident end and an X-ray emitting end;
the X-ray collimator is arranged between the X-ray incident end and the X-ray emitting end; and
the X-ray filter is arranged between the X-ray incident end and the X-ray collimator; or
the X-ray filter is arranged between the X-ray emitting end and the X-ray collimator.

2. The collimator assembly according to claim 1, wherein comprises at least two filters corresponding to different irradiated object sections.

3. The collimator assembly according to claim 2, further comprising a base configured to fix positions of the at least two filters corresponding to different irradiated object sections in the housing, and switch the filters corresponding to different irradiated object sections through the base.

4. The collimator assembly according to claim 3, wherein the base is configured tosh switch the filters corresponding to different irradiated object sections in a push-pull manner.

5. The collimator assembly according to claim 1, wherein the filter has different filtering areas corresponding to different irradiated object sections.

6. The collimator assembly according to claim 1, wherein the filter comprises an opaque heavy metal compound, or the filter comprises a transparent matrix and heavy metal compound particles doped in the transparent matrix.

7. The collimator assembly according to claim 1, wherein there are at least two filters, the filters have different curvatures and correspond to different body parts of a to-be-detected object, and the body parts have different thicknesses in an incident direction of X-rays.

8. The collimator assembly according to claim 1, wherein the filter has a plurality of filtering areas, the filtering areas have different curvatures and correspond to different body parts of the to-be-detected object, and the body parts have different thicknesses in the incident direction of the X-rays.

9. An X-ray source assembly, comprising: a bulb tube, a collimator, and at least one filter; wherein
the filter is located between the X-ray tube and the collimator, or the filter is located on one side of the collimator away from the X-ray tube; and
both the filter and the collimator are located on a path of an X-ray beam generated by the X-ray tube.

10. The X-ray source assembly according to claim 9, wherein the filter has different light filtering areas corresponding to different irradiated object sections.

11. The X-ray source assembly according to claim 9, wherein the filter includes at least two filters corresponding to different irradiated object sections.

12. The X-ray source assembly according to claim 11, further comprising a base configured to fix positions of the at least two filters corresponding to different irradiated object sections, and switch the filters corresponding to different irradiated object sections through the base.

13. The X-ray source assembly according to claim 12, wherein the base is configured to switch the light filters corresponding to different irradiated object sections in a push-pull manner.

14. The X-ray source assembly according to claim 9, wherein the filter comprises a transparent heavy metal compound, or the light filter comprises a transparent matrix and heavy metal compound particles doped in the transparent matrix.

15. The X-ray source assembly according to claim 9, further comprising a mounting structure, the filter being fixed between the collimator and the X-ray tube through the mounting structure, or the filter being fixed to one side of the collimator away from the X-ray tube through the mounting structure.

16. The X-ray source assembly according to claim 15, wherein the mounting structure is located between the collimator and the X-ray tube, and the mounting structure has a first end matching an output end of the X-ray tube, and/or the mounting structure has a second end matching an input end of the collimator, or the mounting structure has no connection with the X-ray tube and/or the light limiter.

17. The X-ray source assembly according to claim 16, wherein thickness of the mounting structure on the path of the X-ray beam is less than a preset threshold.

18. The X-ray source assembly according to claim 15, wherein the mounting structure is located on one side of the collimator away from the X-ray tube, and the mounting structure has a third end matching an output end of the collimator, or the mounting structure has no connection with the collimator.

19. The X-ray source assembly according to claim 9, wherein there are at least two filters, the filters have different curvatures and correspond to different body parts of a to-be-detected object, and the body parts have different thicknesses in an incident direction of X-rays.

20. The X-ray source assembly according to claim 9, wherein the filter has a plurality of light filtering areas, the filtering areas have different curvatures and correspond to different body parts of the to-be-detected object, and the body parts have different thicknesses in the incident direction of the X-rays.
